(19) United States
(12) Patent Application Publication
(10) Pub. No.: US 2004/0192665 A1
(43) Pub. Date: Sep. 30, 2004

(54) Conjugates of porphyrin compounds with chemotherapeutic agents

(75) Inventors: Benjamin Frydman, Madison, WI (US); Aldonia L. Valasinas, Madison, WI (US); John A. Kink, Madison, WI (US); Laurence J. Marton, Fitchburg, WI (US)

Correspondence Address: MORRISON & FOERSTER LLP, 755 PAGE MILL RD, PALO ALTO, CA, 94304-1018, US

(73) Assignee: SLIL BIOMEDICAL CORPORATION, Madison, WI

(21) Appl. No.: 10/627,211

(22) Filed: Jul. 25, 2003

Related U.S. Application Data
(60) Provisional application No. 60/400,512, filed on Aug. 2, 2002.

(51) Int. Cl. A61K 31/555; C07D 487/22; A61K 31/409

(52) U.S. Cl. 514/185; 514/410; 540/145

(57) ABSTRACT

Conjugates of porphyrins with chemotherapeutic agents are disclosed, as well as methods of making the conjugates and methods of treating patients with the conjugates. Porphyrin compounds, such as mesoporphyrin IX, can be covalently linked to chemotherapeutic compounds, such as doxorubicin. The resulting conjugates display decreased systemic toxicity, while preserving the antineoplastic effects of the chemotherapeutic agent. The conjugates are thus useful in treating cancer and other diseases marked by uncontrolled cell proliferation.

### CROSS-REFERENCE TO RELATED APPLICATIONS

This application claims priority benefit of U.S. Provisional Patent Application No. 60/400,512, filed Aug. 2, 2002. The content of that application is incorporated by reference herein in its entirety.

### STATEMENT REGARDING FEDERALLY SPONSORED RESEARCH OR DEVELOPMENT

Not applicable.

### REFERENCE TO AN APPENDIX

Not applicable.

### BACKGROUND OF THE INVENTION

Cancer is the third most common cause of death in the world according to the World Health Organization, after heart disease and infectious disease. Cancer is the second most common cause of death (after heart disease) in the developed world. Accordingly, discovery of new and effective treatments for cancer is a high priority for health care researchers.

Cancer is often treated by using chemotherapy to selectively kill or hinder the growth of cancer cells, while having a less deleterious effect on normal cells. Chemotherapeutic agents often kill rapidly dividing cells, such as cancer cells; non-malignant cells which are dividing less rapidly are affected to a lesser degree. Other agents, such as antibodies attached to toxic agents, have been evaluated for use against cancers. These agents target the cancer cells by making use of a characteristic specific to the cancer, for example, higher-than-normal rates of cell division, or unique antigens expressed on the cancer cell surface.

As toxic agents specifically targeted against cancer cells can enhance therapeutic efficacy, reduce undesirable side effects, or both, many efforts have been made to achieve selective localization of well-defined chemical materials in malignant tumors. A significant advance in the field occurred with the introduction of tetraphenylporphine sulfonates (TPPS), which are non-naturally occurring porphyrins (Winkelman J. (1962) Cancer Res. 22:589). A hematoporphyrin derivative (HPD) was also found to localize in tumors (Lipson R L, Baldes, E J, & Gray M S (1967) Cancer 20: 2255). HPD is a complex mixture of porphyrins currently used as a sensitizer derivative that concentrates in tumor cells and destroys them after the tumor is irradiated with light or a laser beam (Dougherty T J, (1987) Photochem.Photobiol. 45:879). A wide variety of porphyrins and porphyrin analogues have been found to be selectively taken up by tumors, such as the naturally occurring porphyrins; for example, the octacarboxylic uroporphyrins, the tetracarboxylic coproporphyrins, and the dicarboxylic protoporphyrins. Synthetic porphyrins are also selectively taken up by tumors; among them are the meso-tetraphenyl porphyrins and the different porphyrin sulfonates TPPS₄, TPPS₃, TPPS₂ₐ and TPPS₁, which are listed in order of decreasing number of sulfonic acid substituents and decreasing hydrophilicity. Many factors determine the uptake and concentration of porphyrins in the tumors; one important factor is the structure (hydrophobicity, size, polarity) of the compound; another important factor is the formulation in which it is delivered (Sternberg E and Dolphin D (1996) Current Med Chemistry 3, 239). The mechanism(s) of porphyrin localization in tumors is still not entirely clear; the more hydrophobic porphyrins are preferentially incorporated in the lipid core of lipoproteins. Tightly aggregated porphyrins circulate as unbound pseudomicellar structures which can be entrapped in the interstitial regions of the tumor, can be localized in macrophages, or can enter neoplastic cells via pinocytotic processes. Low density lipoproteins (LDL), which are endocytosed by neoplastic cells through a specific receptor-mediated pathway, display the most selective release of porphyrins into the tumors (Jori G (1989) Photosensitizing Compounds, Ciba Foundation Symp 146, pp. 78-94).

The synthesis and cytotoxic actions of porphyrin-polyamine conjugates, and their use in treating diseases such as cancer, have been described in previous patent applications (see International Patent Application Nos. WO 00/66587 and WO 02/10142, U.S. Pat. Nos. 6,392,098, 5,889,061, and 5,677,350, and U.S. Provisional Patent Application No. 60/392,171). These conjugates are taken up by the tumor cells due to their porphyrin moiety, while the polyamine moiety provides the cytotoxic effects. The synthesis and cytotoxic action of certain porphyrin-quinone conjugates have been described in previous patent applications (see International Patent Application No. WO 00/66528 and U.S. patent application Ser. No. 09/562,980.

The current invention describes conjugates of porphyrins with certain chemotherapeutic agents. The conjugates reduce the side effects of the chemotherapeutic agents while maintaining anti-cancer effects of the agents. The conjugates also permit administration of higher doses of chemotherapeutic agents without excessive toxicity or side effects.

### BRIEF SUMMARY OF THE INVENTION

The current invention describes conjugates of porphyrins with chemotherapeutic agents. In another embodiment, the current invention describes conjugates of porphyrins with chemotherapeutic agents, excluding the chemotherapeutic agents of polyamines, polyamine analogs, cyclic polyamines, cyclic polyamine analogs, and quinone compounds. In another embodiment, the current invention describes conjugates of porphyrins with chemotherapeutic agents, excluding the chemotherapeutic agents of polyamines, polyamine analogs, cyclic polyamines, cyclic polyamine analogs, naphthoquinones and naphthoquinone derivatives. In another embodiment, the current invention describes conjugates of porphyrins with chemotherapeutic agents, excluding the chemotherapeutic agents of polyamines, polyamine analogs, cyclic polyamines, cyclic polyamine analogs, dioxonaphthoquinones, hydroxydioxonaphthoquinones, and alkylhydroxydioxonaphthoquinones. In another embodiment, the current invention describes conjugates of porphyrins with chemotherapeutic agents, excluding conjugates of the formula:

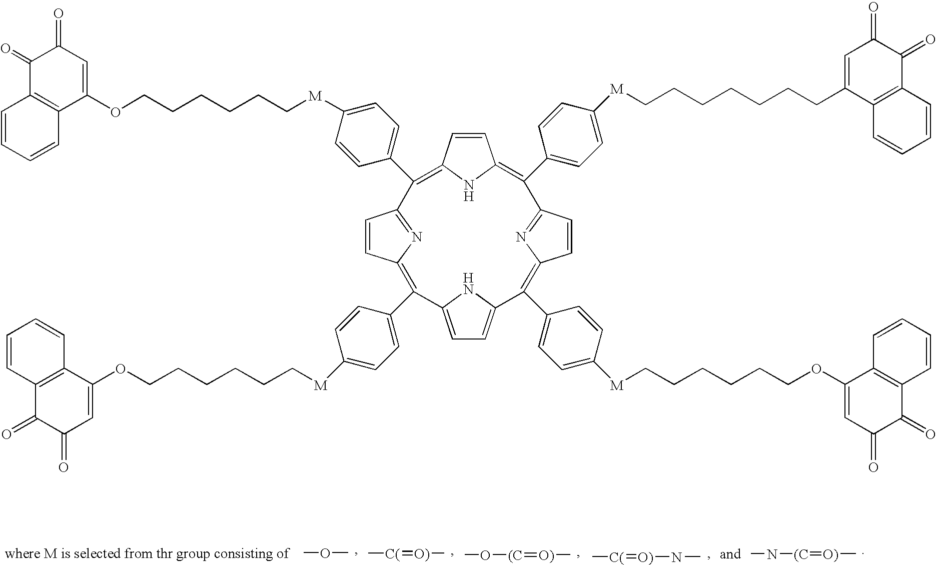

Thus, in one embodiment, the invention embraces a compound comprising a porphyrin and a chemotherapeutic agent, where the chemotherapeutic agent is not a polyamine, polyamine analog, cyclic polyamine, cyclic polyamine analog, dioxonaphthoquinone, or dioxonaphthoquinone derivative, and all salts thereof. In one embodiment, the porphyrin is covalently linked to the chemotherapeutic agent.

In another embodiment of the invention, the porphyrin is selected from the group consisting of mesoporphyrins, deuteroporphyrins, hematoporphyrins, protoporhyrins, uroporphyrins, coproporphyrins, cytoporphyrins, rhodoporphyrin, pyrroporphyrin, etioporphyrins, phylloporphyrins, heptacarboxyporphyrins, hexacarboxyporphyrins, pentacarboxyporphyrins, and other alkylcarboxyporphyrins; and derivatives thereof. In yet another embodiment, the porphyrin is selected from the group consisting of derivatives of deuteroporphyrins. In yet another embodiment, the porphyrin is selected from the group consisting of sulfonic acid derivatives of deuteroporphyrins. In yet another embodiment, the porphyrin is selected from the group consisting of mesoporphyrins. In yet another embodiment, the porphyrin is mesoporphyrin IX.

In another embodiment of the invention, the chemotherapeutic agent is selected from the group consisting of antitumor antibiotics, doxorubicin, bleomycin, dactinomycin, daunorubicin, epirubicin, idarubicin, mitoxantrone, mitomycin, epipodophyllotoxins, etoposide, teniposide, antimicrotubule agents, vinblastine, vincristine, vindesine, vinorelbine, other vinca alkaloids, taxanes, paclitaxel (taxol), docetaxel (taxotere), nitrogen mustards, chlorambucil, cyclophosphamide, estramustine, ifosfamide, mechlorethamine, melphalan; aziridines, thiotepa, alkyl sulfonates, busulfan, nitrosoureas, carmustine, lomustine, and streptozocin, platinum complexes, carboplatin cisplatin, alkylators, altretamine, dacarbazine, procarbazine, temozolamide, folate analogs, methotrexate, purine analogs, fludarabine, mercaptopurine, thiogaunine; adenosine analogs, cladribine, pentostatin, pyrimidine analogs, capecitabine, cytarabine, floxuridine, fluorouracil, gemcitabine, substituted ureas, hydroxyurea, camptothecin analogs, irinotecan, topotecan, topoisomerase I inhibitors, topoisomerase II inhibitors, and anthracycline antibiotics. In another embodiment, the chemotherapeutic agent is doxorubicin.

In yet another embodiment, the chemotherapeutic agent is doxorubicin and the porphyrin is mesoporphyrin IX. In yet another embodiment, the porphyrin-chemotherapeutic agent conjugate is of the structure:

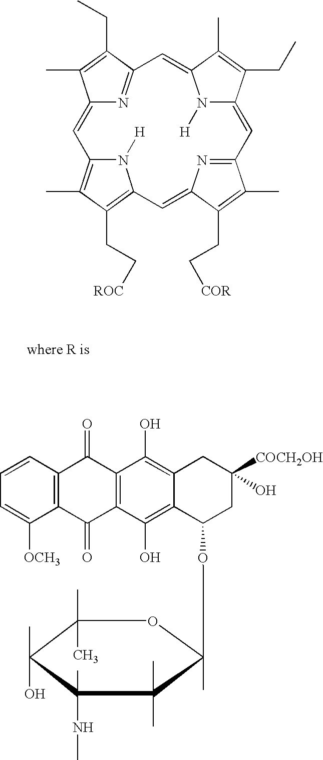

For all of the foregoing compounds, the invention also embraces all stereoisomers, salts, hydrates, and crystalline forms thereof.

The invention also embraces methods of treating a disease, wherein the method comprises administering one or more of the foregoing compounds. The disease can be cancer or any other disease marked by uncontrolled proliferation of cells.

The invention also embraces methods of making the foregoing porphyrin-chemotherapeutic agent conjugates, comprising forming a covalent bond between a porphyrin and a chemotherapeutic agent. In yet another embodiment, the invention embraces a method of making the compound of the structure:

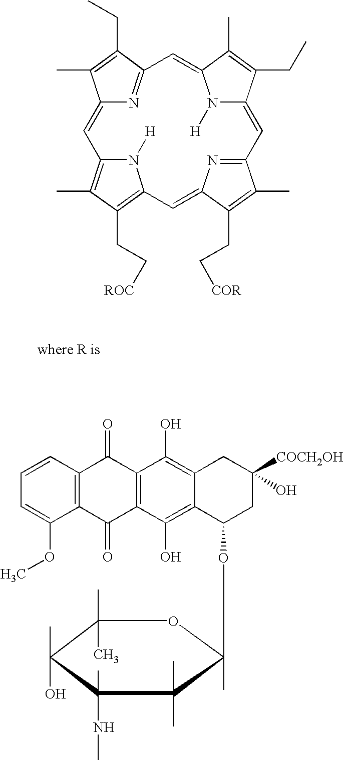

by reacting doxorubicin with mesoporphyrin IX in the presence of a reagent that causes an amide bond to form, where the amide bond is derived from a mesoporphyrin carboxyl group and a doxorubicin amino group.

### BRIEF DESCRIPTION OF THE DRAWINGS

FIG. 1 depicts the synthesis of SL-11180 from mesoporphyrin IX and doxorubicin.

FIG. 2 depicts the effects of SL-11180 administration on the growth of DU-145 tumor cell xenografts in mice.

FIG. 3 depicts the effects of SL-11180 administration on the weight of mice with DU-145 tumor cell xenografts.

FIG. 4 depicts the effects of SL-11180 administration versus doxorubicin administration on the growth of DU-145 tumor cell xenografts in mice.

FIG. 5 depicts the effects of SL-11180 administration versus doxorubicin administration on the weight of mice with DU-145 tumor cell xenografts.

### DETAILED DESCRIPTION OF THE INVENTION

The current invention provides conjugates of porphyrin compounds with chemotherapeutic agents, as well as compositions containing them. In one embodiment, the porphyrin compound is linked to the chemotherapeutic agent by a covalent bond. In another embodiment, the covalent bond can be cleaved in vivo at a rate slow enough to allow accumulation of sufficient porphyrin-chemotherapeutic agent conjugate in the tumor cells, but fast enough to provide free chemotherapeutic agent within the cell to exert a therapeutic effect. In another embodiment, the porphyrin compound is linked to the chemotherapeutic agent by a linking group. In another embodiment, the linking group contains one or more carbon atoms.

In one embodiment, one chemotherapeutic agent is bound to a single porphyrin compound (that is, there is one molecule of chemotherapeutic agent bound to one porphyrin molecule). In one embodiment, one or more chemotherapeutic agents are bound to a single porphyrin compound (that is, there are one or more chemotherapeutic molecules, which can be the same or different molecules, bound to a single porphyrin molecule); for example, two chemotherapeutic agents are bound to a single porphyrin compound. In another embodiment, one or more porphyrins are bound to a single chemotherapeutic agent compound (that is, there are one or more porphyrin molecules, which can be the same or different molecules, bound to a single chemotherapeutic agent molecule). In another embodiment, multiple porphyrins, which can be the same or different molecules, can be bound to multiple chemotherapeutic agents, which can be the same or different molecules, to create a multiple-porphyrin-multiple-chemotherapeutic agent conjugate.

The invention includes all salts of the compounds described herein. In one embodiment, the salts of the compounds comprise pharmaceutically acceptable salts. Pharmaceutically acceptable salts are those salts which retain the biological activity of the free compounds and which are not biologically or otherwise undesirable. The desired salt of a basic compound may be prepared by methods known to those of skill in the art by treating the compound with an acid. Examples of inorganic acids include, but are not limited to, hydrochloric acid, hydrobromic acid, sulfuric acid, nitric acid, and phosphoric acid. Examples of organic acids include, but are not limited to, formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, sulfonic acids, and salicylic acid. Salts of basic compounds with amino acids, such as aspartate salts and glutamate salts, can also be prepared. The desired salt of an acidic compound can be prepared by methods known to those of skill in the art by treating the compound with a base. Examples of inorganic salts of acid compounds include, but are not limited to, alkali metal and alkaline earth salts, such as sodium salts, potassium salts, magnesium salts, and calcium salts; ammonium salts; and aluminum salts. Examples of organic salts of acid compounds include, but are not limited to, procaine, dibenzylamine, N-ethylpiperidine, N,N′-dibenzylethylenediamine, and triethylamine salts. Salts of acidic compounds with amino acids, such lysine salts, can also be prepared.

The invention also includes all stereoisomers of the compounds, including diastereomers and enantiomers, as well as mixtures of stereoisomers, including, but not limited to, racemic mixtures. Unless stereochemistry is explicitly indicated in a structure, the structure is intended to embrace all possible stereoisomers of the compound depicted.

The invention also includes all hydrates of the compounds, and all crystalline forms and non-crystalline forms of the compounds.

The term “alkyl” refers to saturated aliphatic groups including straight-chain, branched-chain, cyclic groups, and combinations thereof, having the number of carbon atoms specified, or if no number is specified, having up to 12 carbon atoms. “Straight-chain alkyl” or “linear alkyl” groups refers to alkyl groups that are neither cyclic nor branched, commonly designated as “n-alkyl” groups. Examples of alkyl groups include, but are not limited to, groups such as methyl, ethyl, n-propyl, isopropyl, butyl, n-butyl, isobutyl, sec-butyl, t-butyl, pentyl, n-pentyl, hexyl, heptyl, octyl, nonyl, decyl, undecyl, dodecyl, neopentyl, cyclopropyl, cyclobutyl, cyclopentyl, cyclohexyl, and adamantyl. Cyclic groups can consist of one ring, including, but not limited to, groups such as cycloheptyl, or multiple fused rings, including, but not limited to, groups such as adamantyl or norbornyl. Preferred subsets of alkyl groups include C₁-C₁₂, C₁-C₁₀, C₁-C₈, C₁-C₆, C₁-C₄, C₁-C₂, C₃-C₄, C₃, and C₄ alkyl groups.

“Substituted alkyl” refers to alkyl groups substituted with one or more substituents including, but not limited to, groups such as halogen (fluoro, chloro, bromo, and iodo), alkoxy, acyloxy, amino, hydroxyl, mercapto, carboxy, benzyloxy, phenyl, benzyl, cyano, nitro, thioalkoxy, carboxaldehyde, carboalkoxy and carboxamide, or a functionality that can be suitably blocked, if necessary for purposes of the invention, with a protecting group. Examples of substituted alkyl groups include, but are not limited to, —CF₃, —CF₂—CF₃, and other perfluoro and perhalo groups.

“Hydroxyalkyl” specifically refers to alkyl groups having the number of carbon atoms specified substituted with one —OH group. Thus, “C₃ linear hydroxyalkyl” refers to —CH₂CH₂CHOH—, —CH₂CHOHCH₂—, and —CHOHCH₂CH₂—.

The term “alkenyl” refers to unsaturated aliphatic groups including straight-chain (linear), branched-chain, cyclic groups, and combinations thereof, having the number of carbon atoms specified, or if no number is specified, having up to 12 carbon atoms, which contain at least one double bond (—C═C—). Examples of alkenyl groups include, but are not limited to, —CH₂—CH═CH—CH₃; and —CH₂—CH₂-cyclohexenyl, where the ethyl group can be attached to the cyclohexenyl moiety at any available carbon valence. The term “alkynyl” refers to unsaturated aliphatic groups including straight-chain (linear), branched-chain, cyclic groups, and combinations thereof, having the number of carbon atoms specified, or if no number is specified, having up to 12 carbon atoms, which contain at least one triple bond (—C≡C—). “Hydrocarbon chain” or “hydrocarbyl” refers to any combination of straight-chain, branched-chain, or cyclic alkyl, alkenyl, or alkynyl groups, and any combination thereof. “Substituted alkenyl,” “substituted alkynyl,” and “substituted hydrocarbon chain” or “substituted hydrocarbyl” refer to the respective group substituted with one or more substituents, including, but not limited to, groups such as halogen, alkoxy, acyloxy, amino, hydroxyl, mercapto, carboxy, benzyloxy, phenyl, benzyl, cyano, nitro, thioalkoxy, carboxaldehyde, carboalkoxy and carboxamide, or a functionality that can be suitably blocked, if necessary for purposes of the invention, with a protecting group.

For all of the foregoing definitions, preferred subsets of the groups include C₁-C₁₂, C₁-C₁₀, C₁-C₈, C₁-C₆, C₁-C₄, C₁-C₂ (when chemically possible), C₃-C₄, C₃, and C₄ groups.

“Aryl” or “Ar” refers to an aromatic carbocyclic group having a single ring (including, but not limited to, groups such as phenyl) or multiple condensed rings (including, but not limited to, groups such as naphthyl or anthryl), and includes both unsubstituted and substituted aryl groups. “Substituted aryls” refers to aryls substituted with one or more substituents, including, but not limited to, groups such as alkyl, alkenyl, alkynyl, hydrocarbon chains, halogen, alkoxy, acyloxy, amino, hydroxyl, mercapto, carboxy, benzyloxy, phenyl, benzyl, cyano, nitro, thioalkoxy, carboxaldehyde, carboalkoxy and carboxamide, or a functionality that can be suitably blocked, if necessary for purposes of the invention, with a protecting group.

“Heteroalkyl,” “heteroalkenyl,” and “heteroalkynyl” refer to alkyl, alkenyl, and alkynyl groups, respectively, that contain the number of carbon atoms specified (or if no number is specified, having up to 12 carbon atoms) which contain one or more heteroatoms as part of the main, branched, or cyclic chains in the group. Heteroatoms include, but are not limited to, N, S, O, and P; N and O are preferred. Heteroalkyl, heteroalkenyl, and heteroalkynyl groups may be attached to the remainder of the molecule either at a heteroatom (if a valence is available) or at a carbon atom. Examples of heteroalkyl groups include, but are not limited to, groups such as —O—CH₃, —CH₂—O—CH₃, —CH₂—CH₂—O—CH₃, —S—CH₂—CH₂—CH₃, —CH₂—CH(CH₃)—S—CH₃, —CH₂—CH₂—NH—CH₂—CH₂—, 1-ethyl-6-propylpiperidino, 2-ethylthiophenyl, and morpholino. Examples of heteroalkenyl groups include, but are not limited to, groups such as —CH═CH—NH—CH(CH₃)—CH₂—. “Heteroaryl” or “HetAr” refers to an aromatic carbocyclic group having a single ring (including, but not limited to, examples such as pyridyl, imidazolyl, thiophene, or furyl) or multiple condensed rings (including, but not limited to, examples such as indolizinyl or benzothienyl) and having at least one hetero atom, including, but not limited to, heteroatoms such as N, O, P, or S, within the ring. Unless otherwise specified, heteroalkyl, heteroalkenyl, heteroalkynyl, and heteroaryl groups have between one and five heteroatoms and between one and twelve carbon atoms. “Substituted heteroalkyl,” “substituted heteroalkenyl,” “substituted heteroalkynyl,” and “substituted heteroaryl” groups refer to heteroalkyl, heteroalkenyl, heteroalkynyl, and heteroaryl groups substituted with one or more substituents, including, but not limited to, groups such as alkyl, alkenyl, alkynyl, benzyl, hydrocarbon chains, halogen, alkoxy, acyloxy, amino, hydroxyl, mercapto, carboxy, benzyloxy, phenyl, benzyl, cyano, nitro, thioalkoxy, carboxaldehyde, carboalkoxy and carboxamide, or a functionality that can be suitably blocked, if necessary for purposes of the invention, with a protecting group. Examples of such substituted heteroalkyl groups include, but are not limited to, piperazine, substituted at a nitrogen or carbon by a phenyl or benzyl group, and attached to the remainder of the molecule by any available valence on a carbon or nitrogen, —NH—SO₂-phenyl, —NH—(C═O)O-alkyl, —NH—(C═O)O-alkyl-aryl, and —NH—(C═O)-alkyl. If chemically possible, the heteroatom(s) as well as the carbon atoms of the group can be substituted. The heteroatom(s) can also be in oxidized form, if chemically possible.

The term “alkylaryl” refers to an alkyl group having the number of carbon atoms designated, appended to one, two, or three aryl groups.

The term “alkoxy” as used herein refers to an alkyl, alkenyl, alkynyl, or hydrocarbon chain linked to an oxygen atom and having the number of carbon atoms specified, or if no number is specified, having up to 12 carbon atoms. Examples of alkoxy groups include, but are not limited to, groups such as methoxy, ethoxy, and t-butoxy.

The term “alkanoate” as used herein refers to an ionized carboxylic acid group, such as acetate (CH₃C(═O)—O⁽⁻¹⁾), propionate (CH₃CH₂C(═O)—O⁽⁻¹⁾), and the like. “Alkyl alkanoate” refers to a carboxylic acid esterified with an alkoxy group, such as ethyl acetate (CH₃C(═O)—O—CH₂CH₃). “ω-haloalkyl alkanoate” refers to an alkyl alkanoate bearing a halogen atom on the alkanoate carbon atom furthest from the carboxyl group; thus, ethyl ω-bromo propionate refers to ethyl 3-bromopropionate, methyl ω-chloro n-butanoate refers to methyl 4-chloro n-butanoate, etc.

The terms “halo” and “halogen” as used herein refer to Cl, Br, F or I substituents.

“Protecting group” refers to a chemical group that exhibits the following characteristics: 1) reacts selectively with the desired functionality in good yield to give a protected substrate that is stable to the projected reactions for which protection is desired; 2) is selectively removable from the protected substrate to yield the desired functionality; and 3) is removable in good yield by reagents compatible with the other functional group(s) present or generated in such projected reactions. Examples of suitable protecting groups can be found in Greene et al. (1991) Protective Groups in Organic Synthesis, 3rd Ed. (John Wiley & Sons, Inc., New York). Amino protecting groups include, but are not limited to, mesitylenesulfonyl (Mts), benzyloxycarbonyl (CBz or Z), t-butyloxycarbonyl (Boc), t-butyldimethylsilyl (TBS or TBDMS), 9-fluorenylmethyloxycarbonyl (Fmoc), tosyl, benzenesulfonyl, 2-pyridyl sulfonyl, or suitable photolabile protecting groups such as 6-nitroveratryloxy carbonyl (Nvoc), nitropiperonyl, pyrenylmethoxycarbonyl, nitrobenzyl, dimethyl dimethoxybenzil, 5-bromo-7-nitroindolinyl, and the like. Hydroxyl protecting groups include, but are not limited to, Fmoc, TBS, photolabile protecting groups (such as nitroveratryl oxymethyl ether (Nvom)), Mom (methoxy methyl ether), and Mem (methoxy ethoxy methyl ether), NPEOC (4-nitrophenethyloxycarbonyl) and NPEOM (4-nitrophenethyloxymethyloxycarbonyl).

“Polyamine analog” is defined as an organic cation structurally similar but non-identical to polyamines such as spermine and/or spermidine and their precursor, diamine putrescine. “Polyamine” is defined as any of a group of aliphatic, straight-chain amines derived biosynthetically from amino acids; several polyamines are reviewed in Marton et al. (1995) Ann. Rev. Pharm. Toxicol. 35:55-91. Polyamines cadaverine and putrescine are diamines produced by decarboxylation of lysine or ornithine, respectively. Putrescine is converted to spermidine, and spermidine to spermine, by the addition of an aminopropyl group. This group is provided by decarboxylated S-adenosyl methionine. Polyamine analogs, which can be branched or un-branched, include, but are not limited to, BE-4444 [1,19-bis(ethylamino)-5,10,15-triazanonadecane]; BE-333 [N1,N11-diethylnorspermine; DENSPM; 1,11-bis(ethylamino)-4,8-diazaundecane; thermine; Warner-Parke-Davis]; BE-33 [N1,N7-bis (ethyl) norspermidine]; BE-34 [N1,N8-bis (ethyl) spermidine]; BE-44 [N1,N9-bis (ethyl) homospermidine]; BE-343 [N1,N12-bis (ethyl) spermine; diethylspermine-N1-N12; DESPM]; BE-373 [N,N′-bis(3-ethylamino) propyl)-1,7-heptane diamine, Merrell-Dow]; BE-444 [N1,N14-bis (ethyl) homospermine; diethylhomospermine-N1-N14]; BE-3443 [1,17-bis (ethylamino)-4,9,14-triazaheptadecane]; BE-4334 [1,17-bis (ethylamino)-5,9,13-triazaheptadecane]; 1,1 2-Me₂-SPM [1,12-dimethylspermine]; various polyamine analogs disclosed in WO 98/17624 and U.S. Pat. No. 5,889,061; and the various novel polyamine analogs disclosed in WO 00/66175 and WO 00/66587, including, but not limited to, compounds designated SL-11027, SL-11028, SL-11029, SL-11033, SL-11034, SL-11037, SL-11038, SL-11043, SL-11044, SL-11047, SL-11048, SL-11050, SL-11090, SL-11091, SL-11092, SL-11093, SL-11094, SL-11098, SL-11099, SL-11100, SL-11101, SL-11102, SL-11103, SL-11104, SL-11105, SL-11108, SL-11114, SL-11118, SL-11119, SL-11121, SL-11122, SL-11123, SL-11124, SL-11126, SL-11127, SL-11128, SL-11129, SL-11130, SL-11132, SL-11133, SL-11134, SL-11136, SL-11137, SL-11141, SL-11144, SL-11150, SL-11201, and SL-11202. Additional polyamine analogs are known in the art, such as O'Sullivan et al. (1997) Bioorg. Med. Chem. 5:2145-2155; and Mukhopadhyaya et al. (1995) Exp. Parasit. 81:39-46; and U.S. Pat. No. 4,935,449.

By “conformationally restricted” is meant that, in a polyamine analog, at least two amino groups are locked or limited in spatial configuration relative to each other. The relative movement of two amino groups can be restricted, for example, by incorporation of a cyclic or unsaturated moiety between adjacent nitrogens (exemplified, but not limited to, a ring, such as a three-carbon ring, four-carbon ring, five-carbon-ring, six-carbon ring, or a double or triple bond, such as a double or triple carbon bond), where the adjacent nitrogens are not included in the conformationally-restricted group. Groups restricting conformational flexibility by means of steric hindrance, yet structurally favorable to the anti-proliferative effects, can also be used for conformational restriction. A “conformationally restricted” polyamine analog can comprise at least two amino groups which are conformationally restricted relative to each other, but can also further comprise amino groups which are not conformationally restricted relative to each other. Flexible molecules such as spermine and BE-444 can have a myriad of conformations and are therefore not conformationally restricted. In both polyamines and polyamine analogs, whether conformationally restricted or not, the amino groups are aliphatic and not aromatic.

Cyclic polyamine compounds and cyclic polyamine analogs are disclosed in International Patent Application WO 02/10142. In certain of these cyclic polyamine compounds, one or more of the aliphatic nitrogens form part of an amide group.

Quinone compounds are compounds which contain a quinone nucleus, such as 1,4-benzoquinone, 1,2-naphthoquinone, or 1,4-naphthoquinone, and derivatives and tautomers thereof. Quinones can be classified by the number of rings they contain; thus, benzoquinones contain only one ring; naphthoquinones contain only two rings; anthraquinones contain only three rings, and so forth. Quinones also include the novel compounds claimed in International Patent Application No. WO 00/66528 and United States Patent Application No. 09/562,980, regardless of the number of rings present in the compounds of that application.

A porphyrin is defined as a compound containing the porphin structure of four pyrrole rings connected by methine or methylene bridges in a cyclic configuration, to which a variety of side chains can optionally be attached. The porphyrin can optionally contain a metal atom or ion. Porphyrin compounds useful in the invention include any porphyrin compound which can be conjugated to a chemotherapeutic agent, preferably via a covalent bond.

Examples of porphyrins which can be used in the invention include (but are not limited to), mesoporphyrins, deuteroporphyrins, hematoporphyrins, protoporhyrins, uroporphyrins, coproporphyrins, cytoporphyrins, rhodoporphyrin, pyrroporphyrin, etioporphyrins, and phylloporphyrins, as well as heptacarboxyporphyrins, hexacarboxyporphyrins, pentacarboxyporphyrins, and other alkylcarboxyporphyrins. Derivatives of the foregoing porphyrins can also be used, including, but not limited to, derivatives of the deuteroporphyrins such as sulfonyl derivatives of deuteroporphyrins (e.g., deuteroporphyrins with one or more sulfonyl or alkylsulfonyl groups on the pyrrole rings). Where structural isomers of a porphyrin class exist, any one of the isomers can be used; for example, any one of mesoporphyrin I, II, III, IV, V, VI, VII, VIII, IX, X, XI, XII, XIII, XIV, or XV can be used, or any one of deuteroporphyrin I-XV, hematoporphyrin I-XV, or protoporphyrin I-XV can be used.

Compounds related to the porphyrins, including, but not limited to, chlorins, bacteriochlorins, chlorophylls, porphyrinogens, phthalocyanines, sapphyrins, corrins, corroles, bilanes, and bilins can also be used in the invention in place of the porphyrin moiety.

Chemotherapeutic agents useful in the invention include any chemical or molecular agent administered for chemotherapy; that is, any chemical or molecular agent which can be used to treat a disease caused by uncontrolled proliferation of cells, such as cancer. In one embodiment, the chemotherapeutic agents exclude polyamines, polyamine analogs, cyclic polyamines, cyclic polyamine analogs, and quinone compounds. In another embodiment, the chemotherapeutic agents exclude polyamines, polyamine analogs, cyclic polyamines, cyclic polyamine analogs, and dioxonaphthoquinone and dioxonaphthoquinone derivative compounds.

General classes of, and specific examples of, chemotherapeutic agents useful in the invention include (but are not limited to):

antitumor antibiotics, such as doxorubicin, bleomycin, dactinomycin, daunorubicin, epirubicin, idarubicin, mitoxantrone, and mitomycin;

epipodophyllotoxins such as etoposide and teniposide;

antimicrotubule agents, such as vinblastine, vincristine, vindesine, vinorelbine, and other vinca alkaloids;

taxanes, such as paclitaxel (taxol) and docetaxel (taxotere);

nitrogen mustards, such as chlorambucil, cyclophosphamide, estramustine, ifosfamide, mechlorethamine, and melphalan;

aziridines such as thiotepa;

alkyl sulfonates such as busulfan;

nitrosoureas such as carmustine, lomustine, and streptozocin;

platinum complexes such as carboplatin and cisplatin;

alkylators such as altretamine, dacarbazine, procarbazine, and temozolamide;

folate analogs such as methotrexate;

purine analogs such as fludarabine, mercaptopurine, and thiogaunine;

adenosine analogs such as cladribine and pentostatin;

pyrimidine analogs such as capecitabine, cytarabine, floxuridine, fluorouracil, and gemcitabine;

substituted ureas such as hydroxyurea;

camptothecin analogs such as irinotecan and topotecan;

topoisomerase inhibitors, such as topoisomerase I inhibitors (e.g. camptothecin) and topoisomerase II inhibitors (e.g. doxorubicin, daunorubicin, etoposide, amsacrine, and mitoxantrone);

anthracycline antibiotics such as doxorubicin;

and any other chemotherapeutic agent which can be covalently conjugated to a porphyrin moiety.

Conjugation of the porphyrin to the chemotherapeutic agent can be accomplished by chemical cross-linking methods well known in the art. For example, to conjugate a carboxylic acid-containing porphyrin, such as a mesoporphyrin (e.g. mesoporphyrin IX) or coproporphyrin (e.g. coproporphyrin I), to a chemotherapeutic agent containing an amino group, well-known condensation agents can be used. These agents include, but are not limited to, carbodiimides (e.g., dicyclohexylcarbodiimide, diisopropylcarbodiimide, 1-ethyl-3-(3-dimethylaminopropyl)-carbodiimide (EDC)) or onium reagents (onium salts, e.g., (benzotriazol-1-yloxy)tris(dimethylamino)phosphonium hexafluorophosphate (BOP), O-(7-azabenzotriazol-1-yl)-N,N,N′,N′-tetramethyluronium hexafluorophosphate (HATU), O-(benzotriazol-1-yl)-N,N,N′,N′-tetramethyluronium hexafluorophosphate (HBTU), or O-(Benzotriazol-1-yl)-N,N,N′,N′-tetramethyluronium tetrafluoroborate (TBTU)). Other methods, such as converting the carboxylic acid function of the porphyrin into an acid chloride, an active ester derivative (e.g., an N-hydroxysuccinimide active ester derivative), or otherwise activating the carboxylic acid group to nucleophilic attack, can be used. These condensation reactions can also be used for forming ester bonds between carboxylic acid-containing porphyrins and hydroxy-containing chemotherapy agents.

Cross-linking agents can also be used to link porphyrins to chemotherapeutic agents. References such as Wong, Shan S., Chemistry of protein conjugation and cross-linking, CRC Press: Boca Raton, 1991, detail reactive groups and linking groups suitable for cross-linking porphyrins with chemotherapeutic agents. Linkers can contain a moiety reactive with the porphyrins and a second moiety reactive with the chemotherapeutic agent. For example, a compound such as sulfosuccinimidyl 4-(N-maleimidomethyl)cyclohexane-1-carboxylate (sulfo-SMCC), which is available commercially, can be used to link an amine-containing porphyrin with a thiol-containing chemotherapeutic agent. A wide variety of linkers can be used, and the invention is not limited by the type of linker used. Examples of linkers include, but are not limited to, substituted and unsubstituted C₁-C₁₂ alkyl, alkenyl, and alkynyl groups, C₁-C₁₂ heteroalkyl, heteroalkenyl, and hetereoalkynyl groups, and C₆-C₂₀ aryl-containing and heteroaryl-containing linking groups.

For porphyrins, such as the etioporphyrins, which do not contain an activatable group, the porphyrin-chemotherapeutic agent conjugate can be formed either by non-covalent association, or appropriate derivatization of the porphyrin itself. For example, etioporphyrins bearing halogens on their alkyl side chains can be synthesized (see, e.g., Bauder, C et al.; Synlett (6), 335-7 (1990); Yon-Hin, P et al.; Can. J. Chem. 68(10), 1867-75 (1990); Clewlow, P J et al.; J. Chem. Soc., Perkin Trans. 1 (7), 1925-36 (1990); and Clewlow, P J et al.; J. Chem. Soc., Chem. Commun. (11), 724-6 (1985)); the halogenated etioporphyrin can then be reacted with an appropriate nucleophile. The nucleophile can contain a second reactive group (with a protecting group if necessary) that can then be reacted with the chemotherapeutic agent to form the conjugate; alternatively, the chemotherapeutic agent itself can be the nucleophile.

Therapeutic Use of Porphyrin-Chemotherapeutic Agent Conjugates

Porphyrin-chemotherapeutic agent conjugates of the present invention are useful for treatment of a variety of diseases caused by uncontrolled proliferation of cells, including cancer, such as prostate cancer. The compounds are used to treat mammals, preferably humans. “Treating” a disease using a porphyrin-chemotherapeutic agent conjugate of the invention is defined as administering one or more porphyrin-chemotherapeutic agent conjugates of the invention, with or without additional therapeutic agents, in order to prevent, reduce, or eliminate either the disease or the symptoms of the disease, or to retard the progression of the disease or of symptoms of the disease. “Therapeutic use” of the porphyrin-chemotherapeutic agent conjugates of the invention is defined as using one or more porphyrin-chemotherapeutic agent conjugates of the invention to treat a disease, as defined above.

In order to evaluate the efficacy of a particular porphyrin-chemotherapeutic agent conjugate for a particular medicinal application, the compounds can be first tested against appropriately chosen test cells in vitro. In a non-limiting example, porphyrin-chemotherapeutic agent conjugates can be tested against tumor cells, for example, prostate tumor cells. Exemplary experiments can utilize cell lines capable of growing in culture as well as in vivo in athymic nude mice, such as LNCaP (see Horoszewicz et al. (1983) Cancer Res. 43:1809-1818). Culturing and treatment of carcinoma cell lines, cell cycle and cell death determinations based on flow cytometry are described in the art, for example, Mi et al. (1998) Prostate 34:51-60; Kramer et al. (1997) Cancer Res. 57:5521-27; and Kramer et al. (1995) J. Biol. Chem. 270:2124-2132. Evaluations can also be made of the effects of the porphyrin-chemotherapeutic agent conjugate on cell growth and metabolism.

Analysis can begin with IC₅₀ determinations based on dose-response curves ranging from 0.1 to 1000 μM performed at 72 hr. From these studies, conditions can be defined which produce about 50% growth inhibition and used to: (a) follow time-dependence of growth inhibition for up to 6 days, with particular attention to decreases in cell number, which may indicate drug-induced cell death; (b) characterize porphyrin-chemotherapeutic agent conjugate effects on cell cycle progression and cell death using flow cytometry (analysis to be performed on attached and detached cells); (c) examine porphyrin-chemotherapeutic agent conjugate effects on cellular metabolic parameters. Porphyrin-chemotherapeutic agent conjugate effects can be normalized to intracellular concentrations (by HPLC analysis), which also provide an indication of their relative ability to penetrate cells.

In Vivo Testing of Porphyrin-Chemotherapeutic Agent Conjugates

Porphyrin-chemotherapeutic agent conjugates found to have potent anti-proliferative activity in vitro towards cultured carcinoma cells can be evaluated in in vivo model systems. The first goal is to determine the relative toxicity of the compounds in non-tumor-bearing animals, such as DBA/2 mice. Groups of three animals each can be injected intraperitoneally with increasing concentrations of a porphyrin-chemotherapeutic agent conjugate, beginning at, for example, 10 mg/kg. Toxicity as indicated by morbidity is closely monitored over the first 24 hr. The toxicity of the porphyrin-chemotherapeutic agent conjugate can also be tested versus the free chemotherapeutic agent, that is, versus the same chemotherapeutic agent which is present in the porphyrin-chemotherapeutic agent conjugate but without a conjugated porphyrin.

After the highest tolerated dosage is deduced, antitumor activity is determined. Typically, tumors can be subcutaneously implanted into nude athymic mice by trocar and allowed to reach 100-200 mm³ before initiating treatment by intraperitoneal injection, for example on a schedule of daily×5 d. Porphyrin-chemotherapeutic agent conjugates can be given in a range between, for example, 10 and 200 mg/kg. Porphyrin-chemotherapeutic agent conjugates can be evaluated at three treatment dosages with 10-15 animals per group (a minimum of three from each can be used for pharmacodynamic studies, described below). Mice can be monitored and weighed twice weekly to determine tumor size and toxicity. Tumor size is determined by multi-directional measurement from which volume in mm³ is calculated. Tumors can be followed until median tumor volume of each group reaches 1500 mm³ (i.e., 20% of body weight), at which time the animals can be sacrificed. The initial anti-tumor studies can focus on a bolus dosing schedule, such as daily×5 d schedule; however, constant infusion can be performed via Alzet pump delivery for 5 days since this schedule can lead to increased efficacy (see Sharma et al. (1997) Clin. Cancer Res. 3:1239-1244). In addition to assessing anti-tumor activity, free porphyrin-chemotherapeutic agent conjugate levels and free chemotherapeutic agent levels in tumor and normal tissues can be determined in test animals.

Methods of Administration of Porphyrin-Chemotherapeutic Agent Conjugates

The porphyrin-chemotherapeutic agent conjugates of the present invention can be administered to a mammalian, preferably human, subject via any route known in the art, including, but not limited to, those disclosed herein. Methods of administration include but are not limited to, oral, intravenous, intraarterial, intratumoral, intramuscular, topical, inhalation, subcutaneous, intraperitoneal, gastrointestinal, and directly to a specific or affected organ. Oral administration in particular is a convenient route for administration and is a preferred route of administration, particularly when oral administration provides equivalent therapeutic results as compared with other routes. The porphyrin-chemotherapeutic agent conjugates of the invention are well-tolerated orally and chemotherapeutic agents which ordinarily could not be administered orally, or which could not be administered orally in sufficient amounts, can be successfully administered in therapeutically effective amounts as part of the porphyrin-chemotherapeutic agent conjugates. The porphyrin-chemotherapeutic agent conjugates described herein are administratable in the form of tablets, pills, powder mixtures, capsules, granules, injectables, creams, solutions, suppositories, emulsions, dispersions, food premixes, and in other suitable forms. The compounds can also be administered in liposome formulations. The compounds can also be administered as prodrugs, where the prodrug undergoes transformation in the treated subject to a form which is therapeutically effective. Additional methods of administration are known in the art.

The pharmaceutical dosage form which contains the compounds described herein is conveniently admixed with a non-toxic pharmaceutical organic carrier or a non-toxic pharmaceutical inorganic carrier. Typical pharmaceutically-acceptable carriers include, for example, mannitol, urea, dextrans, lactose, potato and maize starches, magnesium stearate, talc, vegetable oils, polyalkylene glycols, ethyl cellulose, poly(vinylpyrrolidone), calcium carbonate, ethyl oleate, isopropyl myristate, benzyl benzoate, sodium carbonate, gelatin, potassium carbonate, silicic acid, and other conventionally employed acceptable carriers. The pharmaceutical dosage form can also contain non-toxic auxiliary substances such as emulsifying, preserving, or wetting agents, and the like. A suitable carrier is one which does not cause an intolerable side effect, but which allows the novel porphyrin-chemotherapeutic agent conjugate(s) to retain its pharmacological activity in the body. Formulations for parenteral and nonparenteral drug delivery are known in the art and are set forth in Remington's Pharmaceutical Sciences, 18th Edition, Mack Publishing (1990). Solid forms, such as tablets, capsules and powders, can be fabricated using conventional tableting and capsule-filling machinery, which is well known in the art. Solid dosage forms, including tablets and capsules for oral administration in unit dose presentation form, can contain any number of additional non-active ingredients known to the art, including such conventional additives as excipients; desiccants; colorants; binding agents, for example syrup, acacia, gelatin, sorbitol, tragacanth, or polyvinylpyrollidone; fillers, for example lactose, sugar, maize-starch, calcium phosphate, sorbitol or glycine; tableting lubricants, for example magnesium stearate, talc, polyethylene glycol or silica; disintegrants, for example potato starch; or acceptable wetting agents such as sodium lauryl sulfate. The tablets can be coated according to methods well known in standard pharmaceutical practice. Liquid forms for ingestion can be formulated using known liquid carriers, including aqueous and non-aqueous carriers, suspensions, oil-in-water and/or water-in-oil emulsions, and the like. Liquid formulations can also contain any number of additional non-active ingredients, including colorants, fragrance, flavorings, viscosity modifiers, preservatives, stabilizers, and the like. For parenteral administration, porphyrin-chemotherapeutic agent conjugates can be administered as injectable dosages of a solution or suspension of the compound in a physiologically acceptable diluent or sterile liquid carrier such as water or oil, with or without additional surfactants or adjuvants. An illustrative list of carrier oils would include animal and vegetable oils (e.g., peanut oil, soy bean oil), petroleum-derived oils (e.g., mineral oil), and synthetic oils. In general, for injectable unit doses, water, saline, aqueous dextrose and related sugar solutions, and ethanol and glycol solutions such as propylene glycol or polyethylene glycol are preferred liquid carriers. The pharmaceutical unit dosage chosen is preferably fabricated and administered to provide a final concentration of drug at the point of contact with the cancer cell of from, for example, 1 μM to 10 mM or from, for example, 1 to 100 μM. The optimal effective concentration of porphyrin-chemotherapeutic agent conjugates can be determined empirically and will depend on the type and severity of the disease, route of administration, disease progression and health and mass or body area of the patient. Such determinations are within the capability of one skilled in the art. Porphyrin-chemotherapeutic agent conjugates can be administered as the sole active ingredient, or can be administered in combination with another active ingredient, including, but not limited to, cytotoxic agents, antibiotics, antimetabolites, polypeptides, antibodies, cytokines, or one or more chemotherapeutic agents which are not conjugated to porphyrins.

### EXAMPLE 1

Synthesis of a Porphyrin-Doxorubicin Conjugate

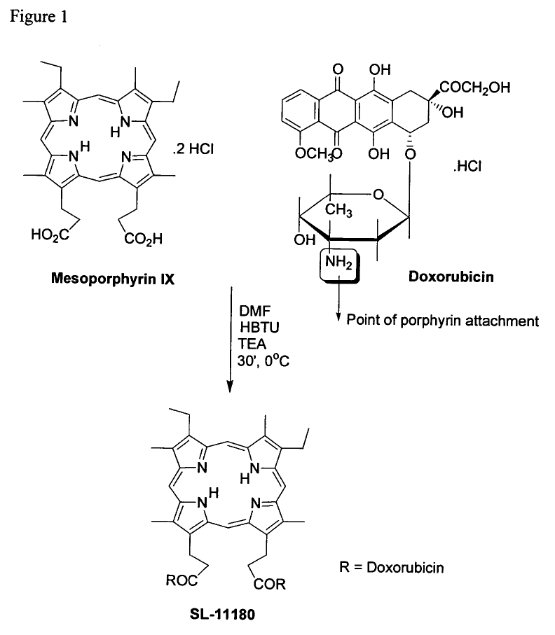

The synthesis (see FIG. 1) is performed according to the following overall reaction:

Mesoporphyrin IX.2HCl (MW=639)+Doxorubicin.HCl (MW=580)→SLIL-11180 (MW1616).

Doxorubicin (520 mg, 0.89 mmol), mesoporphyrin IX.2HCl (286 mg, 0.44 mmol) and triethylamine (0.51 ml, 3.56 mmol) were dissolved in dimethylformamide (30 ml), cooled to 5° C. under nitrogen with constant stirring, and HBTU (337 mg, 0.89 mmol) was added. The mixture was kept for a further ½ hour, the solvent removed in vacuo, and the residue dissolved in chloroform, washed with a saturated solution of sodium chloride (twice), dried and evaporated. The residue was purified by chromatography through a silica gel column using chloroform: methanol/9:1 as eluant. After evaporation of the solvent, the product was crystallized from chloroform:methanol 9:1/hexane (v/v); 595 mg (82%) of the conjugate was obtained. MALDI-MS (m/z): 1617.6 (M⁺+H), 1693.5 (M⁺+Na), 1222.6,1204.6. HPLC: column: 4.6×250 mm C18 VYDAC SN 910401, 300 Angstrom pores, particles 5 micron; Eluant A=0.1% Trifluoroacetic acid (TFA); Eluant B=90% Acetonitrile in 0.008% TFA; Eluant B increases at the rate of 2%/min. Rt: 50.53 min (95% potency).

Note that in the depiction in FIG. 1, the Haworth convention is used to draw the daunosamine moiety of doxorubicin.

### EXAMPLE 2

SL-11180 (Porphyrin-Doxorubicin Conjugate) Effectively Treats DU-145 Xenografts in Nude Mice

In order to determine whether SL-11180 (porphyrin-doxorubicin conjugate, (P/D)) is effective against prostate cancer, a well-characterized nude mouse xenograft model using DU-145 human prostate tumor cells was utilized. This model is used extensively to predict the efficacy of experimental drugs in human cancer patients.

This example involves: (a) description of the DU-145 xenograft tumor model; (b) treatment with SL-11180 via different dosing routes; and (c) comparison of efficacy between SL-11180 and doxorubicin.

(a) Male, 5-6 week old nude mice (nu/nu) were purchased from Harlan Sprague-Dawley (Madison, Wis.) and acclimated in the laboratory for at least 1 week prior to experimentation. The animals were housed in micro-isolator cages, at 5-7 animals per cage. The mice were maintained on a 12-hour light/dark cycle and received autoclaved rodent food and water. Cage were cleaned and bedding changed once weekly. Irradiated corn cob bedding was used. Animals were observed daily and clinical signs were noted.

Hormonal non-responsive prostate tumor cell line, DU-145 (American Type Cell collection, ATCC, MD) was maintained in liquid culture prior to injection into the mice. DU-145 cells were grown in culture flasks with Dulbecco's modified Eagle media (DMEM) (Gibco, Grand Island, N.Y.) containing 5% fetal bovine serum. The adherent DU-145 cells were recovered from the flasks using trypsin (0.05%)/EDTA (0.53 mM) (Gibco) and harvested by low-speed centrifugation (1000-1200×g). The cells were resuspended at 10⁷/ml in DMEM. Each mouse was injected sub-cutaneously (S.Q.) with 10⁶ DU-145 in 100 ul in the right rear flank using a 27 gauge needle and syringe. The tumors were allowed to grow and reach a palpable tumor size of approximately 5-10 mm³ before the start of the treatment. This tumor volume was typically reached within 10 to 15 days post-injection. Animals were divided into the various treatment groups to give an overall equivalent average tumor volume for each group. Tumor size was measured twice per week in two perpendicular dimensions with a vernier caliper and converted to tumor volume using the formula: (l×w²)/2, where l and w refer to the longer and the shorter dimensions, respectively. Animal body weights were taken twice per week at the same time as the tumors were measured. Morbidity and mortality were monitored daily.

SL-11180 treatments were initiated approximately 15 days after DU-145 tumor cell injection. SL-11180 was formulated in a delivery vehicle consisting of 25% DMSO, 35% glycerol and 40% distilled de-ionized water. The drug was administered at 100 to 200 mg/kg (depending on the route of administration) to each mouse once per week for 5 weeks. The dosage level was determined by exact body weight. Mice treated with delivery vehicle administered intraperitonally, (I.P.), served as a placebo control.

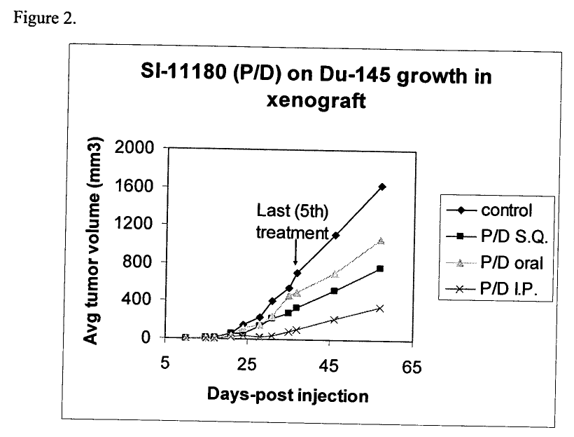

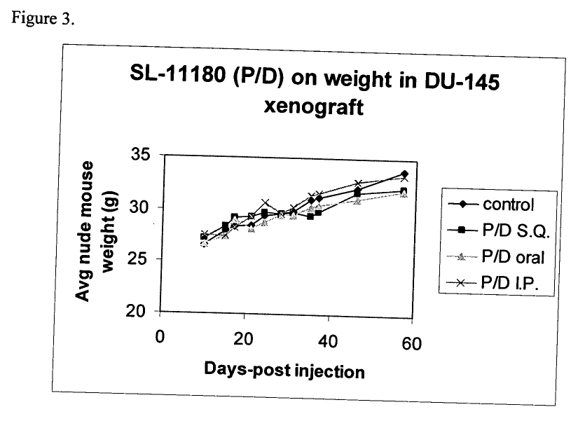

(b) In experiment 1, the efficacy of the SL-11180 in the tumor model to placebo was compared. SL-11180 was administered via 3 different routes; either I.P., oral, or S.Q. routes. Five mice per treatment group were tested. A dosage of 100 mg/kg (once weekly) was administered in the I.P. and the S.Q. treated groups using a 27 gauge needle. The oral treated group received SL-11180 at 200 mg/kg (once weekly) using an 18 gauge feeding needle (Popper and Sons, New Hyde Park, N.Y.). The efficacy of SL-111180 against DU-145 in vivo and the effect of the drug on total body weight are shown in FIGS. 2 and 3, respectively.

The results shown in FIG. 2 strongly indicates that treatment with SL-11180 can inhibit tumor growth in this model. Compared to the treatment control, all three treatment routes (I.P., oral, S.Q.) showed a significant reduction in tumor volume. SL-11180 administered by I.P. at 100 mg/kg showed the most dramatic effect with up to a 10-fold reduction in tumor volume up to day 31. In the SL-11180 treated animals, average tumor volume at this time was 39 mm , whereas the tumor volume in the placebo-treated groups was 405 mm^{3.} Lower, but significant inhibition of tumor growth of 5 to 6-fold was seen after about day 31. SL-11180 administered by the oral and S.Q. routes both showed intermediate efficacy with an overall 2-fold reduction in tumor volume compared to placebo controls. Lower efficacy seen in the S.Q. treated animals compared to I.P. administration may have been due to deposition of SL-11180 at the S.Q. administration site. The reduced efficacy by oral treatment is probably due to reduced bioavailability of SL-11180.

As shown in FIG. 3, the SL-11180 delivered at the doses at all three administration routes showed no overt toxicity in the mice as measured by body weight. No overt morbidity or mortality was noted and all the treated animals appeared healthy. Moreover, all mice treated with SL-11180 steadily increased their body weight by 15-20%, consistent with the placebo controls. The only observation worth noting is that there was an obvious accumulation of drug deposited at the injection site in the S.Q. treated group. The deposition of drug did not appear to affect the health of the animal, but may have hindered its ability to reach the tumor.

(c) The efficacy of SL-11180 against DU-145 xenografts was compared to doxorubicin, a widely used anti-neoplastic agent known to be effective in this model. Six to seven nude mice per group were each injected with a DU-145 at 10⁶ cells per mouse. Once the xenografts were established, the mice were treated I.P. with either SL-11180 at 120 mg/kg or 8 mg/kg of doxorubicin. The dose of 8 mg/kg of doxorubicin is an often published, high-end dose for cancer therapy in vivo. SL-11180 was prepared as described above in a DMSO/glycerol/water delivery vehicle and doxorubicin-hydrochloride (Calbiochem, La Jolla, Calif.) was prepared in water. Mice treated I.P. with the delivery vehicle served as the placebo controls. Animals were treated once per week for 5 weeks.

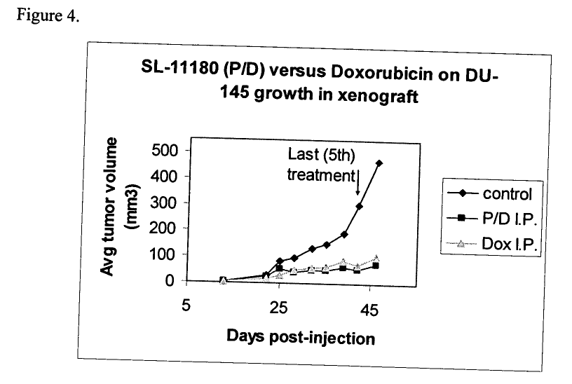

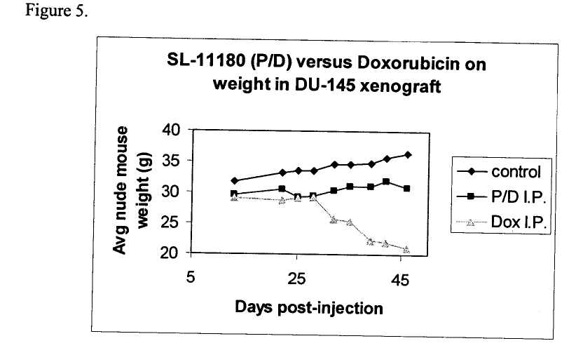

The ability to inhibit growth of DU-145 in xenograft by SL-11180 compared to doxorubicin is shown in FIG. 4. Tumor volumes were, on average, 6-fold less in the animals after 5 treatments with SL-11180 compared to the placebo-control tumors. Average tumor volume in the placebo control group 46 days after injection was 470 mm³, while tumor volume in the SL-111180 treated group was 74 mm^{3.} Tumor volumes were 4.4-fold less after 5 treatments with doxorubicin. The average tumor volume in this group at this time was 106 mm^{3.} This experiment confirms the ability of SL-11180 to effectively inhibit the growth of DU-145 in xenografts as found in the first experiment. Moreover, it indicates the SL-11180 may be more effective than doxorubicin. As shown below in FIG. 5, the greater inhibition of tumor growth by SL-11180 compared to doxorubicin may be far more significant because of its reduced toxicity.

The toxicity in nude mice of SL-11180 compared to doxorubicin, as determined by body weight is shown in FIG. 5. As expected, the placebo control treated animals steadily gained weight over time with no toxicity. Significantly, the SL-11180 treated mice at 120 mg/kg dose administered I.P. showed no overt toxicity. No overt morbidity was noted and all the treated animals appeared healthy and maintained body weight with no weight loss (FIG. 5). This agrees with the results in the first experiment were mice treated at a slightly lower dose (100 mg/kg) gained weight. In contrast, mice treated with doxorubicin showed significant toxic side-effects as manifested by severe weight loss and death. As shown in FIG. 5 after the second treatment on day 25, all animals in the doxorubicin treated mice began to lose weight. After the 5^{th} treatment, average group weight in the surviving animals was down by 27%. Only half the animals (3/6) survived after 5 treatments with doxorubicin at 8 mg/kg. In that group one mouse died several days after the 3^{rd}, 4^{th} and 5^{th} treatments. The severe toxicity by doxorubicin at this dose probably accounts in part for its ability to inhibit tumor growth (FIG. 4). Lower treatment doses of doxorubicin should improve toxicity, but as a consequence would decrease its ability to inhibit tumor growth.

These experiments indicate that SL-11180, a porphyrin-doxorubicin conjugate, administered systemically by I.P. is an effective therapeutic against prostate cancer in vivo. Furthermore, as judged by safety and efficacy, SL-11180 is a superior drug compared to doxorubicin. The conjugate of porphyrin with doxorubicin (SL-11180) is much less toxic than doxorubicin alone, but the potent anti-cancer properties of doxorubicin is maintained. This reduced toxicity of SL-11180 is believed to be due to its improved targeting to the cancer cell by porphyrin.

All references, publications, patents and patent applications mentioned herein are hereby incorporated by reference herein in their entirety.

Although the foregoing invention has been described in some detail by way of illustration and example for purposes of clarity and understanding, it will be apparent to those skilled in the art that certain changes and modifications may be practical. Therefore, the description and examples should not be construed as limiting the scope of the invention, which is delineated by the appended claims.

1. A compound comprising:
a porphyrin, and
a chemotherapeutic agent,
wherein said chemotherapeutic agent is not a polyamine, polyamine analog, cyclic polyamine, cyclic polyamine analog, dioxonaphthoquinone, or dioxonaphthoquinone derivative;
and all salts, hydrates, crystalline forms, and stereoisomers thereof.

2. The compound of claim 1, wherein the porphyrin is covalently linked to the chemotherapeutic agent.

3. The compound of claim 2, wherein the porphyrin is covalently linked to the chemotherapeutic agent via a linking group.

4. The compound of claim 2, wherein the porphyrin is selected from the group consisting of mesoporphyrins, deuteroporphyrins, hematoporphyrins, protoporhyrins, uroporphyrins, coproporphyrins, cytoporphyrins, rhodoporphyrin, pyrroporphyrin, etioporphyrins, phylloporphyrins, heptacarboxyporphyrins, hexacarboxyporphyrins, pentacarboxyporphyrins, and other alkylcarboxyporphyrins; and derivatives thereof.

5. The compound of claim 4, wherein the porphyrin is selected from the group consisting of derivatives of deuteroporphyrins.

6. The compound of claim 5, wherein the porphyrin is selected from the group consisting of sulfonic acid derivatives of deuteroporphyrins.

7. The compound of claim 4, wherein the porphyrin is a mesoporphyrin.

8. The compound of claim 7, wherein the porphyrin is mesoporphyrin IX.

9. The compound of claim 2, wherein the chemotherapeutic agent is selected from the group consisting of antitumor antibiotics, doxorubicin, bleomycin, dactinomycin, daunorubicin, epirubicin, idarubicin, mitoxantrone, mitomycin, epipodophyllotoxins, etoposide, teniposide, antimicrotubule agents, vinblastine, vincristine, vindesine, vinorelbine, other vinca alkaloids, taxanes, paclitaxel (taxol), docetaxel (taxotere), nitrogen mustards, chlorambucil, cyclophosphamide, estramustine, ifosfamide, mechlorethamine, melphalan, aziridines, thiotepa, alkyl sulfonates, busulfan, nitrosoureas, carmustine, lomustine, and streptozocin, platinum complexes, carboplatin cisplatin, alkylators, altretamine, dacarbazine, procarbazine, temozolamide, folate analogs, methotrexate, purine analogs, fludarabine, mercaptopurine, thiogaunine, adenosine analogs, cladribine, pentostatin, pyrimidine analogs, capecitabine, cytarabine, floxuridine, fluorouracil, gemcitabine, substituted ureas, hydroxyurea, camptothecin analogs, irinotecan and topotecan, topoisomerase I inhibitors, topoisomerase II inhibitors, and anthracycline antibiotics.

10. The compound of claim 2, wherein the chemotherapeutic agent is doxorubicin.

11. The compound of claim 2, wherein the chemotherapeutic agent is doxorubicin and the porphyrin is mesoporphyrin IX.

12. The compound of claim 11 of the structure:

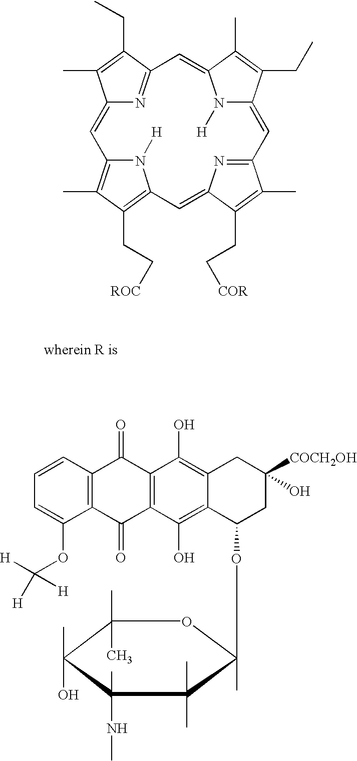

13. A method of treating a disease characterized by uncontrolled cell proliferation, wherein the method comprises administering a therapeutically effective amount of a compound of claim 2.

14. The method of claim 13, wherein the disease is cancer.

15. A method of treating a disease characterized by uncontrolled cell proliferation, wherein the method comprises administering a therapeutically effective amount of the compound of claim 10.

16. A method of making a compound of claim 2, comprising forming a covalent bond between a porphyrin and a chemotherapeutic agent.

17. A method of making the compound of claim 12, comprising reacting doxorubicin with mesoporphyrin IX in the presence of a reagent that causes an amide bond to form, said amide bond form by reaction of a mesoporphyrin carboxyl group and a doxorubicin amino group.

18. The method of claim 17, wherein the reagent that causes an amide bond to form is selected from the group consisting of onium reagents and carbodiimides.
